(19) United States
(12) Patent Application Publication
(10) Pub. No.: US 2026/0263628 A1
(43) Pub. Date: Sep. 10, 2026

(54) AN ORAL LIQUID COMPOSITION OF VINORELBINE

(71) Applicants: INSTITUT GUSTAVE ROUSSY, VILLEJUIF (FR); UNIVERSITE PARIS-SACLAY, GIF-SUR-YVETTE (FR); CENTRE NATIONAL DE LA RECHERCHE SCIENTIFIQUE, PARIS (FR)

(72) Inventors: MAXIME ANNEREAU, VINCENNES (FR); ANTOINE DOWEK, PARIS (FR); FRANÇOIS-XAVIER LEGRAND, ORSAY (FR); ANDRÉ RIEUTORD, PARIS (FR)

(21) Appl. No.: 19/118,401

(22) PCT Filed: Oct. 5, 2023
(86) PCT No.: PCT/EP2023/077540

§ 371 Date: Apr. 4, 2025

(30) Foreign Application Priority Data
Oct. 5, 2022 (EP) 22306494.0

(51) Int. Cl. A61K 47/69; A61K 9/00; A61K 9/08; A61K 31/475; A61K 47/12; A61K 47/26

(52) U.S. Cl. CPC A61K 47/6951; A61K 9/0053; A61K 9/08; A61K 31/475; A61K 47/12; A61K 47/26

(57) ABSTRACT

The invention provides a pharmaceutical composition in oral liquid form, comprising vinorelbine, or a pharmaceutically acceptable salt thereof, and a cyclodextrin that is sulfobutylether-beta-cyclodextrin (SBE-beta-CD), preferably wherein at least 95%, vinorelbine, or said pharmaceutically acceptable salt thereof, is in form of an inclusion complex with said cyclodextrin.

The present invention relates to an oral liquid form of vinorelbine, particularly useful for oral administration to children.

### BACKGROUND OF THE INVENTION

In pediatrics, vinorelbine is used in the maintenance phase for the treatment of rhabdomyosarcomas (RMS). RMS are malignant soft tissue tumors, which account for 60 to 70% of soft tissue cancers in children. The median age of diagnosis is about 5 years.

Vinorelbine is a semi-synthetic antimitotic agent belonging to the vinca alkaloid family. The anti-tumor activity of this antineoplastic is mediated by the inhibition of tubulin polymerization which induces a blockage of mitosis in G2-M phase and thus leads to cell death. The raw material vinorelbine tartrate is an amorphous white hydrophilic powder that absorbs moisture from the air. Carcinogenic, mutagenic and reprotoxic, this powder is also irritant and can be fatal if inhaled, ingested or absorbed. It is totally soluble in water, partially soluble in methanol and insoluble in aprotic solvents.

Vinorelbine is currently available as an injectable form of vinorelbine ditartrate at 10 mg/ml or soft capsules at 20 and 30 mg for oral administration. Each intravenous administration requires a day hospitalization and represents a particularly constraining management with an impact on the quality of life of the patients. The choice of the oral route simplifies patient management, increases comfort by reducing the number of weekly hospitalizations required by the protocol for intravenous administration of vinorelbine. However solid oral forms may represent an obstacle for young children and adolescents with swallowing difficulties. Furthermore factors such as capsule rupture, dissolution of the active ingredient and gastric emptying have an important impact on the absorption of oral forms of vinorelbine resulting in large inter-individual variations in pharmacokinetics (PK) (Zhou et al, Biopharm Drug Dispos 1994; 15:577-86). A French national multicenter phase II trial (OviMa-1210) to evaluate the efficacy and PK of oral vinorelbine in patients aged from 6 to 17 years with recurrent or progressive unresectable low-grade glioma demonstrated lower drug exposure (in half of the cases) in children due to a shorter elimination half-life suggesting that higher doses may be required for pediatric patients (Hamimed et al. Br J Clin Pharmacol 2022; 88:2096-117). This study shows that the large variability in PK is due to a combination of variability in apparent PK parameters during oral administration in children: altered bioavailability due to physiological processes affecting intestinal absorption of drugs in pediatrics, affected volume of distribution, and higher clearance.

In vinorelbine oral administration, cases of mucositis and oropharyngeal toxicity have been reported in the institution: adverse reaction linked to the vesicant propriety of vinorelbine which, on contact with the mucous membranes, triggers an inflammatory reaction responsible for irritation, burning and even necrosis (AFSOS. Mucositis and candidiasis 2015; HUG. Chemotherapy extravasation: management procedure 2019).

Thus, the existing oral forms for pediatric use cause mucosal toxicities due to vesicant property of vinorelbine, lead to variability in pharmacokinetics and are not adapted to the whole pediatric population.

There is thus a need to provide improved oral forms of vinorelbine in terms of size, control of pharmacokinetics and adverse effects.

### SUMMARY OF THE INVENTION

The present invention provides an oral form of vinorelbine that is suitable with pediatric use and overcomes problems of oropharyngeal toxicity and of variations of pharmacokinetics, while also providing a taste-masking effect.

More particularly, the invention provides a pharmaceutical composition in oral liquid form, comprising vinorelbine, or a pharmaceutically acceptable salt thereof, and a cyclodextrin that is sulfobutylether-beta-cyclodextrin (SBE-beta-CD). In a preferred embodiment, the composition comprises an aqueous solution of vinorelbine, or a pharmaceutically acceptable salt thereof, forming an inclusion complex with said cyclodextrin. Preferably at least 90%, or preferably at least 95%, of vinorelbine, or of the pharmaceutically acceptable salt thereof, is in form of an inclusion complex with said cyclodextrin.

The invention further provides an inclusion complex comprising vinorelbine, or a pharmaceutically acceptable salt thereof, and a cyclodextrin that is sulfobutylether-beta-cyclodextrin (SBE-beta-CD), preferably wherein the molar ratio of vinorelbine to the cyclodextrin is of 1:2 to 1:5.

A solution, preferably an aqueous solution, which comprises such inclusion complex is also provided, preferably wherein at least 90%, still preferably at least 95%, vinorelbine, or said pharmaceutically acceptable salt thereof, is in form of said inclusion complex with the cyclodextrin.

The pharmaceutical composition may be a ready-to-use solution or may be a reconstituted form. A solid composition, preferably a lyophilized composition, suitable for reconstitution, which composition has been obtained from the solution described herein is thus also provided.

### BRIEF DESCRIPTION OF THE FIGURES

FIG. 1 shows complexation curves of vinorelbine with SGM or with SBE-beta-CD to reach concentration of 20 mg/mL vinorelbine. To obtain 95% of complexation for a solution at 20 mg/mL (ie 25.7 mmol/L), about 62 mmol/L of SGM or about 71 mmol/L of SBE-beta-CD is needed.

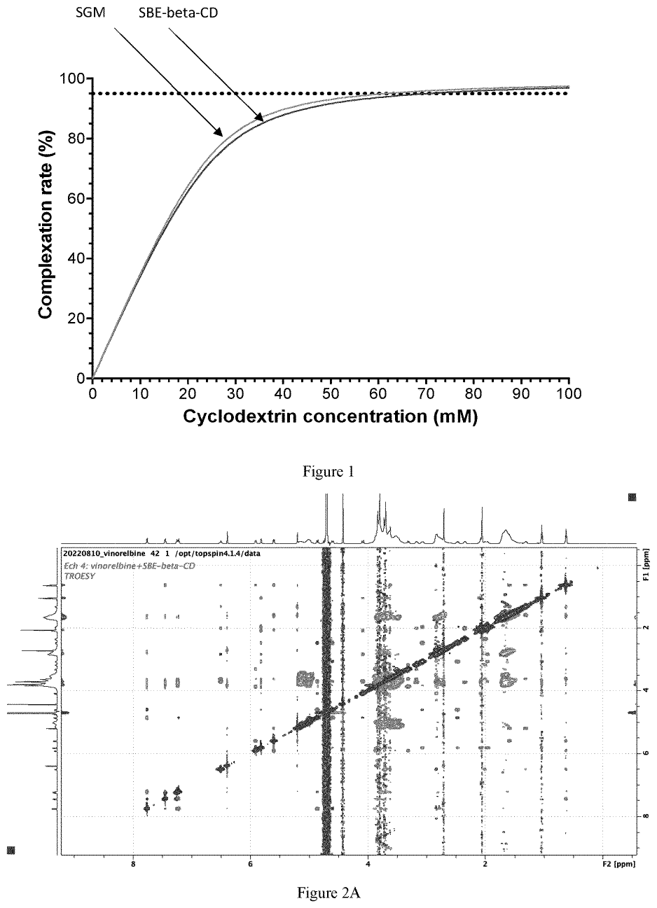

FIGS. 2A to 2D show the 2D off-resonance ROESY NMR spectrum of the inclusion complex comprising SBE-beta-CD and vinorelbine. FIG. 2A is the full contour map of the 2D off-resonance ROESY NMR spectrum and FIGS. 2C to 2D are expansions of regions of interest. Attributions of SBE-beta-CD protons and interacting protons of vinorelbine are labelled on the indirect and the direct dimensions respectively.

FIG. 3 shows a representative curve of viability following tissue contact with vinorelbine alone and vinorelbine in complexation with SBE-beta-CD.

FIG. 4 shows histological images after hematoxylin-eosin-saffron (HES) staining (top), Ki67 immunostaining (middle top), TUNEL staining (middle bottom) and E-cadherin immunostaining (bottom).

### DETAILED DESCRIPTION OF THE INVENTION

Throughout the present description, unless otherwise indicated, the term “vinorelbine” will include its different forms such as pharmaceutically acceptable salts and/or solvates thereof.

A “salt” of vinorelbine includes the acid addition salts and base addition salts thereof. Suitable acid addition salts are formed from acids which form non-toxic salts. Suitable base salts are formed from bases which form non-toxic salts. Preferred pharmaceutically acceptable salts include sulfate, acetate and ditartrate salts. Tartrate salt is highly preferred.

A “solvate” is used herein to describe a molecular complex comprising the compound and one or more pharmaceutically acceptable solvent molecules, for example water. The term “hydrate” refers to a solvate, when the solvent is water.

The vinorelbine ditartrate salt hydrate is according to formula (I) below.

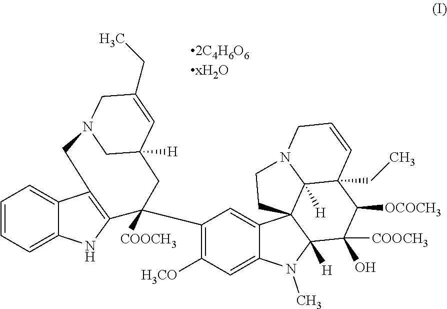

Vinorelbine ditartrate is also known as Navelbine®.

Cyclodextrins (CD) are cyclic oligosaccharides with hydroxyl groups on the outer surface and a void cavity in the center. Their outer surface is hydrophilic, and therefore they are usually soluble in water, but the cavity has a lipophilic character. The most common cyclodextrins are alpha-cyclodextrin, beta-cyclodextrin and gamma-cyclodextrin, consisting of 6, 7 and 8 alpha-1,4-linked glucose units, respectively. The number of these units determines the size of the cavity.

The inventors have found that sulfobutylether-beta-cyclodextrin (SBE-beta-CD) forms stable inclusion complexes with vinorelbine. The encapsulation protects against mucosal toxicity.

The term “inclusion complex” refers to a chemical complex in which one chemical compound (the “host”) has a cavity into which a “guest” compound can be accommodated. The interaction between the host and guest involves purely van der Waals bonding. In the present description, the host of the inclusion complex is the cyclodextrin, and the guest is vinorelbine or a pharmaceutically acceptable salt and/or solvate thereof, preferably vinorelbine ditartrate hydrate.

In a particular embodiment, SBE-beta-CD forms inclusion complexes by interacting with vinorelbine at protons at positions 11′, 12′, 13′ and 14′ as well as protons at positions 20′, 21′, 3′ and 5′ of vinorelbine.

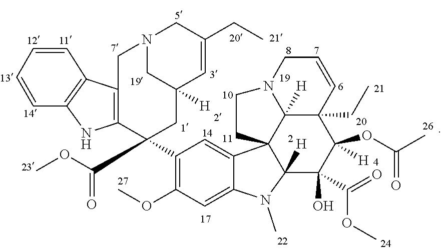

SBE-beta-CD comprises a macrocyclic ring of 7 glucose subunits, wherein at least part of the outer hydroxy groups of the macrocyclic ring are substituted with sulfobutyl ether groups.

The number of sulfobutyl ether substitutions per macrocyclic ring may vary in a wide range depending among others on the desired rate of complexation. One skilled in the art is able to adapt the conditions for the synthesis of SBE-beta-CD so as to obtain the desired number of sulfobutyl ether substitutions. Preferably, the SBE-beta-CD is of the chemical formula (II) below, wherein each R(1a-f), R(2a-f), R(3a-f) is independently —OH or —O—CH₂—CH₂—CH₂—CH₂—SO₃H, optionally in the form of a sodium salt (SO₃Na).

The average number of sulfobutyl ether substitutions per cyclodextrin molecule may range between 3 and 10, preferably between 5.5 and 7.5 or between 6.2 and 6.9.

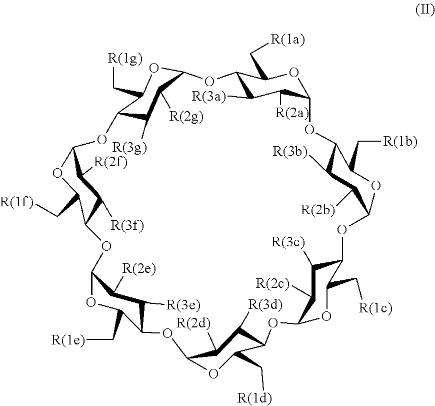

A sulfoalkyl ether cyclodextrin is typically ionized at physiological pH and is therefore accompanied by a counter ion, such as Nat. In the present description, the term “sulfobutylether-beta-cyclodextrin” encompasses both the sulfonic acid (SO₃H) and the salt forms thereof, such as the sodium sulfonate (SO₃Na) form thereof. Preferably, each sulfonic acid group of the SBE-beta-CD is in a sodium sulfonate (SO₃Na) form. The term “sulfobutylether-beta-cyclodextrin” also encompasses mixtures of sulfobutylether-beta-cyclodextrin with different numbers of sulfobutyl ether substitutions.

The sodium salt of SBE-beta-CD is also known as betadex sulfobutyl ether sodium and is, e.g., sold under the tradenames Captisol™ and Advasep 7™ by Ligand Pharmaceuticals and Dexolve™ by Cyclolab.

In one embodiment, the sulfobutyl substitution is specifically defined in the sulfobutyl-bet-cyclodextrin used according to the invention. For instance, the sulfobutyl-bet-cyclodextrin may comprise exactly 1, 2, 3, 4, 5, 6 or 7 sulfobutyl substitutions per cyclodextrin molecule.

Advantageously, the molar ratio of vinorelbine to the cyclodextrin may be 1:2 to 1:5, preferably 1:4 to 1:5, preferably about 1:4.

In one embodiment, the pharmaceutical composition is a liquid composition. Preferably, said composition is a solution. Preferably, said composition is not a suspension. In a preferred embodiment, the solvent is water.

In one embodiment, the composition of the present invention is in the form of a concentrate. Within this application, a “concentrate” is referred to as a formulation which preferably is not administered directly to a patient but diluted before use. For example, the concentrate can be diluted with a suitable liquid, e.g. water, alternatively with 5 percent glucose or sucralose solutions or saline, to give a ready-for-use formulation. Alternatively, the concentrate may be used directly.

In one embodiment, the concentration of vinorelbine or any pharmaceutically acceptable salt thereof is in the range from about 1 mg/mL to about 40 mg/mL. In a preferred embodiment, said concentration is in the range from about 20 mg/mL. In particular, amounts (i.e. mg/mL) refer to an amount of vinorelbine [i.e. in the free form], and if a salt thereof (e.g., tartrate salt) is used, the amount will be adapted accordingly (e.g. 27.67 mg/mL ditartrate).

In one embodiment, the concentration of vinorelbine or any pharmaceutically acceptable salt thereof is in the range from about 1 percent to about 10 percent (w/v). In a preferred embodiment, said concentration is about 2 percent (w/v).

In one embodiment, the concentration of SBE-beta-CD or sodium salt thereof is in the range of about 2.5 mmol/L to about 250 mmol/L, preferably in the range of about 25 mmol/L to about 125 mmol/L, more preferably about 100 mmol/L.

In one embodiment, the concentration of cyclodextrin is in the range of 0.1 percent to 70 percent (w/v). In a preferred embodiment, said concentration is in the range of 2 percent to 25 percent (w/v). In another preferred embodiment, said concentration is in the range of 2 percent to 20 percent (w/v). In a more preferred embodiment, said concentration is about 17.5 percent (w/v).

In one embodiment, the pH of the composition is in the range of 3-8. In a preferred embodiment, the pH of the composition is about 4.

In a preferred embodiment, the pharmaceutical composition comprises vinorelbine or a pharmaceutically acceptable salt thereof such as tartrate salt in a concentration of 1 mg/mL to 40 mg/mL, the cyclodextrin is in a concentration in the range of 2 percent to 20 percent (w/v), and the pH of the composition is about 4. In particular, amounts (i.e. mg/mL) refer to an amount of vinorelbine [i.e. in the free form], and if a salt thereof (e.g., tartrate salt) is used, the amount will be adapted accordingly.

In a preferred embodiment, the pharmaceutical composition comprises vinorelbine or a pharmaceutically acceptable salt thereof such as tartrate salt in a concentration of about 1 percent to about 10 percent (w/v), and the SBE-beta-CD in a concentration of about 2% to about 25% (w/v).

The composition may further comprise at least one taste enhancing or masking agent. Taste enhancing or masking agents are organolaeptic additives used for improvement of taste. It can be a sweetener, for example, sodium saccharin, sucrose, glucose, fructose, aspartame, potassium acesulfame and/or sucralose, in a concentration range of 0.05-1 percent (w/v). In a preferred embodiment, said taste enhancing/masking agent is sucralose, preferably with a concentration of 0.5 percent (w/v).

In one embodiment, the composition may further comprise at least one flavouring agent, i.e. a flavour enhancer.

In a preferred embodiment, the pharmaceutical composition comprises vinorelbine or a pharmaceutically acceptable salt thereof in a concentration of about 2 percent (w/v), SBE-beta-CD in a concentration of about 15.2 percent (w/v) and sucralose in a concentration of 0.5 percent (w/v). Preferably, the pH of the composition is in the range of about 4.

In an embodiment, in the pharmaceutical composition of the invention, at least 90%, preferably at least at least 95%, more preferably at least 98%, of the vinorelbine is complexed with the SBE-beta-CD. That means that the composition comprises less than 10%, preferably less than 5%, in particular less than 2%, free (not complexed) vinorelbine.

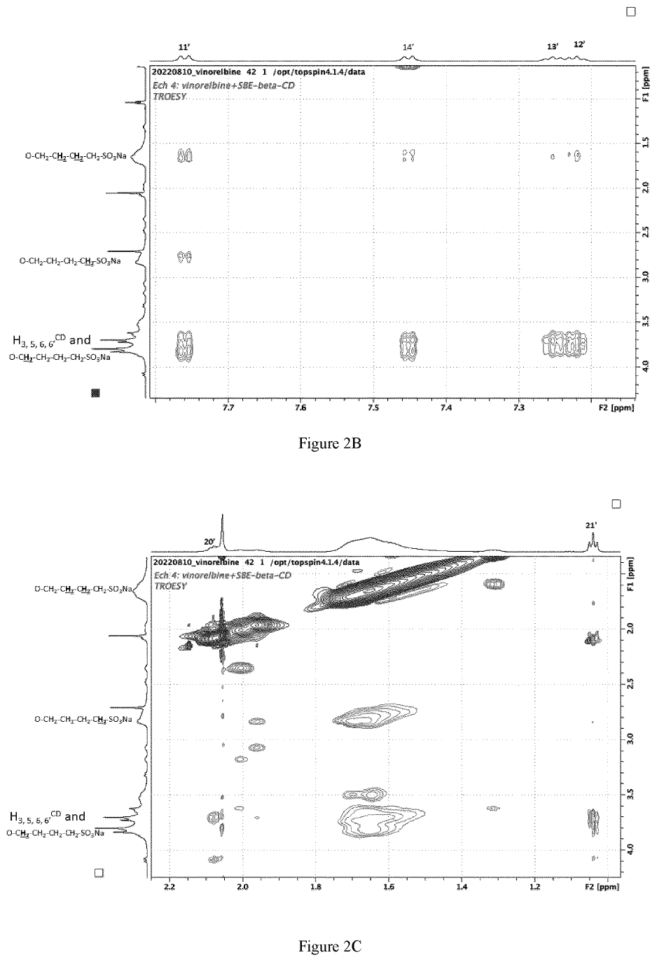

The inclusion complex formed by vinorelbine and SBE-beta-CD is also an object of the present invention. Said complex preferably presents the NMR characterization as presented in FIGS. 2A-2D.

In one embodiment, the pharmaceutical composition is free or substantially free of preservatives.

The pharmaceutical composition of the invention may further comprise one or more agents that reduce the rate by which the active ingredient will decompose. Such agents, which are referred to herein as “stabilizers,” include, but are not limited to, antioxidants such as citric or ascorbic acid, pH buffers, or salt buffers, etc.

One or more pharmaceutically acceptable pH adjusting agents and/or buffering agents can be included in a composition of the invention, including acids such as acetic, boric, citric, lactic, phosphoric and hydrochloric acids; bases such as sodium hydroxide, sodium borate, sodium citrate, sodium acetate, sodium lactate and trishydroxymethylaminomethane; and buffers such as citrate/dextrose, sodium bicarbonate and ammonium chloride. Such acids, bases and buffers are included in an amount required to maintain pH of the composition.

Preferably, the pharmaceutical composition of the invention provides a complexation of vinorelbine with SBE-beta-CD of sufficient high affinity for the taste-masking effect to be maintained and the oro-pharyngeal toxicity to be avoided during oral administration, and a complexation of sufficiently low affinity for vinorelbine to be released from SBE-beta-CD when in the stomach.

The invention also provides methods of preparing a liquid formulation.

A first method comprises the steps of: forming a first aqueous solution comprising said cyclodextrin; forming a suspension comprising active agent (i.e. vinorelbine or a pharmaceutically acceptable salt thereof); and mixing said solution and suspension to form the liquid formulation.

A second method is similar to the first step except that vinorelbine or a pharmaceutically acceptable salt thereof is added directly to the first solution without formation of the suspension.

A third method is similar to the first except that the cyclodextrin and/or cyclodextrin derivative is added directly to the suspension without formation of the first solution.

A fourth method comprises the steps of: adding a suspension comprising vinorelbine or a pharmaceutically acceptable salt thereof to a powdered or particulate cyclodextrin and/or cyclodextrin derivative.

A fifth method comprises the steps of: adding vinorelbine or a pharmaceutically acceptable salt thereof directly to the powdered or particulate cyclodextrin and/or cyclodextrin derivative; and adding a second solution.

A sixth method comprises the steps of: creating the liquid formulation by any of the above methods and then isolating a solid material by lyophilisation, spray-drying, spray-freeze-drying, antisolvent precipitation, a process utilizing a supercritical or near supercritical fluid, or other methods known to those of ordinary skill in the art to make a powder for reconstitution.

A liquid formulation of the invention may also be converted to a solid formulation for reconstitution. A reconstitutable solid pharmaceutical composition according to the invention comprises vinorelbine or a pharmaceutically acceptable salt thereof, said cyclodextrin and optionally at least one other pharmaceutical excipient. This composition is reconstituted with an aqueous liquid to form a liquid formulation that is preserved. The composition can comprise an admixture of a solid derivatized cyclodextrin and vinorelbine-containing solid and optionally at least one solid pharmaceutical excipient, such that a major portion of the active agent is not complexed with the derivatized cyclodextrin prior to reconstitution. Alternatively, the composition can comprise a solid mixture of said cyclodextrin and vinorelbine or a pharmaceutically acceptable salt thereof, wherein a major portion of the active agent is complexed with the derivatized cyclodextrin prior to reconstitution.

The reconstitutable formulation can be prepared according to any of the following processes. A liquid formulation of the invention is first prepared, then a solid is formed by lyophilization (freeze-drying), spray-drying, spray freeze-drying, antisolvent precipitation, various processes utilizing supercritical or near supercritical fluids, or other methods known to those of ordinary skill in the art to make a solid for reconstitution.

The pharmaceutical composition is useful for oral administration in subjects in need thereof.

The subject to treat may be any human patient, including children, adolescents, adults. In one embodiment, the subject is a subject who may have difficulty swallowing a solid oral form, e.g. children or the elderly. The term “child” or “children” as used herein refers to children not more than 12 years of age, and includes children from about 12 months to about 12 years of age. Adolescents of 12-18 years old are also encompassed.

The regimen can be adjusted by the physician, and will depend on the disease and the subject. Typically the pharmaceutical composition may be administered at a dose of 60-80 mg/m2, e.g. once a week. Metronomic administrations are also encompasses. Instead of using short bursts of the maximum tolerated dose chemotherapy interspersed with long breaks to allow recovery from the harmful side effects, a dose therapy involving the frequent, even daily, oral administration of vinorelbine at doses significantly below the maximum tolerated dose, without prolonged drug-free breaks. An example of such low dosage would be e.g. 1-10 mg/day.

It is herein described methods for treating cancers by oral administration of the pharmaceutical composition of vinorelbine that is herein provided. Cancers include, for example, breast tumors, non-small cell lung tumors, rhabdomyosarcomas (RMS), as well as gliomas, such as optic and pontine gliomas.

The Examples illustrate the invention without limiting its scope.

### EXAMPLES

### Example 1: Selection of Cyclodextrins Suitable for Complexing Vinorelbine

### Materials and Methods

### Choice of Cyclodextrins: Isothermal Titration Calorimetry

In the first instance, 12 CDs and CD derivatives supplied by Captisol® (Kansas, USA), SIGMA® (St Louis, Missouri, USA) and MSD® (New Jersey, USA) and authorized by the FDA or the European Medicines Agency (EMA) were tested (U.S Food and Drug Administration C for DE and. Food Additive Status List. FDA 2022. U.S Food and Drug Administration C for DE and. Select Committee on GRAS Substances 2022. Committee for Human Medicinal Products. Cyclodextrins used as excipients. EMA 2017:16).

These were alpha-CD, beta-CD, CRYSMEB (methyl-beta-CD, low substitution degree), RAMEB (methyl-beta-CD, higher substitution degree), DIMEB (di-O-methyl-beta-CD), TRIMEB (tri-O-methyl-beta-CD), HP-beta-CD (Hydroxypropyl-beta-CD), SBE-beta-CD (Sulfobutylether-beta-CD), gamma-CD, HP-gamma-CD (Hydroxypropyl-gamma-CD), SBE-gamma-CD (Sulfobutylether-gamma-CD), and sugammadex (SGM). Vinorelbine ditartrate and 50 mM CD solutions were prepared in phosphate buffer pH 7.0. For each system, a titration experiment was performed by filling the sample cell and syringe with 202.8 μL of a concentrated 0.5 mM degassed vinorelbine solution and a 5 mM CD solution, respectively. The formation constants and inclusion enthalpies were determined simultaneously for each CD/vinorelbine system using an isothermal calorimeter (ITC200, MicroCal Inc., USA) [34-36]. To confirm the validity of the chosen stoichiometric model and to increase the accuracy of the thermodynamic parameters, each system was also subjected to a competition experiment with SGM: a solution of 0.5 mM vinorelbine and 10 mM CD titrated against a mixture containing 5 mM SGM and 10 mM CD. For all tests, after addition of an initial 0.5 μL aliquot, 10 aliquots of 3.5 μL of the syringe solution were delivered over 7 s for each injection with a time interval between two consecutive injections of 70-90 s that proved sufficient for a systematic and complete return to baseline. For all CDs the measurements were performed at 25° C., and the stirring speed was set at 1000 rpm. The resulting heat flux was recorded as a function of time. Prior to data analysis, blank titrations were performed under the same experimental conditions by injecting individual species into buffer, and/or buffer into species and buffer into buffer to determine the heat of dilution. These values were subtracted from the heats measured in the presence of vinorelbine and CD. The peak area after each injection was obtained by integration of the resulting signal and was expressed as the heat effect per injection. The binding constant (K_{B}), inclusion enthalpy (Δ_{b}H°), and heat capacity (Δ_{b}Cp®) were determined using a program developed by Bertaut and Landy involving a global analysis of all binding isotherms obtained for a given system (Bertaut et al. Beilstein J Org Chem 2014:12).

A set of thermodynamic parameters was employed for simultaneous nonlinear regression analyses of titration and competition experiments.

### Choice of Cyclodextrins: 2D NMR

In order to confirm the type of existing interaction (inclusion complex, type of binding) between CD and vinorelbine, a two-dimensional proton NMR was performed on PA (Spitzer et al, J Heterocycl Chem 1995; 32:1255-9), CDs (Schneider et al, Chem Rev 1998; 98:1755-86) and complexes (Vogt et al, Mol Pharm 2012; 9:3357-74; Péan et al, J Chim Phys Phys-Chim Biol 1999; 96:1486-93). Vinorelbine 10 mM stock solution (SS) was prepared by dissolving 21.58 mg of vinorelbine tartrate in 2 mL of deuterated water. CD stock solutions were prepared by dissolving: 21.78 mg SGM in 1 mL heavy water (SS2), 9.73 mg alpha-CD in ImL heavy water (SS3), 21.63 mg SBE-beta-CD in 1 mL heavy water (SS4), and 20.72 mg SBE-gamma-CD in 1 mL heavy water (SS5). These stock solutions were used for the preparation of 10 samples containing vinorelbine, CDs or a mixture of CDs and vinorelbine in deuterated water as shown in Table 1.

The samples were analyzed using a Bruker® Ascend™ spectrometer composed of a superconducting magnet allowing the application of a 700 MHz magnetic field. Data acquisition and processing is performed with the Bruker TopSpin 4.0.B software. On all the samples, a 1D spectrum of the proton was performed in order to obtain characteristic lines allowing to define the position, the chemical shift, the amplitude as well as the short distance couplings of the protons. In order to highlight the spatial proximity of certain protons in the vinorelbine-CD complex, samples containing vinorelbine were subjected to 2D dipolar correlation experiments (ROESY off resonance: rotating frame Overhauser effect spectroscopy). FIGS. 2A-2D present ROESY spectrum of the sample comprising the vinorelbine-SBE-beta-CD complex. To obtain more detailed information, protons (vinorelbine and CD) were attributed by COSY (correlated spectroscopy).

### Results

The titration experiments allowed to determine the formation of a bond between vinorelbine and CD by measuring the heat variation. No heat variation was observed in the tests with native CDs except for alpha-CD which gave a small heat variation. In total, out of 12 CDs tested, only 4 interacted with vinorelbine. The CD derivatives: SGM, SBE-beta-CD, SBE-gamma-CD and alpha-CD are those for which an interaction is observed, then the competition experiments with sugammadex allowed to determine the binding constants. The results obtained are summarized in Table 2.

During sample preparation for 2D NMR, a haze appeared in sample 2 when vinorelbine was added to the vial containing SGM. A range of vinorelbine-SGM mixtures was performed from 1.5 mmol/L to 5 mmol/L. The haze appears from the mixture of concentration 2.5 mmol/L (sample 10).

Using the constant values of Table 2, we simulated the complexation of vinorelbine in CD depending on the CD concentration and the final concentration of the vinorelbine solution. To achieve a concentration of 10 mg/mL, approximately 49 mmol/L sugammadex or 58 mmol/L SBE-beta-CD is required to reach 95% of complexation. For 18 mg/mL, approximately 59 mmol/L sugammadex or 68 mmol/L SBE-beta-CD is required to obtain the same complexation rate. 2D NMR showed that vinorelbine interacts with protons at positions 11′, 12′, 13′ and 14′ as well as protons at positions 20′ and 21′. This indicates a real and strong complexation between vinorelbine and cyclodextrin.

### DISCUSSION

For a positive enthalpy variation, the constant increases with the temperature increase, whereas it decreases with the temperature increase for a negative enthalpy variation. In this context, CDs with a positive enthalpy variation and/or a high binding constant are preferred especially since the body temperature is higher than the working temperature. From there arises the question of the dissociation of the complex in the organism. The interaction being dependent on the protonation of the compounds, the pH of the solution is another important parameter for the establishment of the bond. From the results obtained at 25° C., SBE-beta-CD appears to be more advantageous with an enthalpy variation favorable to complexation, a high association constant. Moreover, contrary to sugammadex, SBE-beta-CD does not produce any precipitate when mixed with vinorelbine which is compatible with the solution formulation.

TABLE 1
Sample preparation for 2D NMR

| | | | | | | Heavy |
|---|---|---|---|---|---|---|
| | | Vinorelbine | CD | Vinorelbine | CD SS | water |
| | | concentration | concentration | SS volume | volume | volume |
| Sample | CD | (mM) | (mM) | (μL) | (μL) | (μL) |
| 1 | — | 5 | — | 300 | — | 300 |
| 2 | SGM | 5 | 5 | 300 | 300 | — |
| 3 | SGM | — | 5 | — | 300 | 300 |
| 4 | SBE-beta- | 5 | 5 | 300 | 300 | — |
| | CD | | | | | |
| 5 | SBE-beta- | — | 5 | — | 300 | 300 |
| | CD | | | | | |
| 6 | SBE- | 5 | 5 | 300 | 300 | — |
| | gamma-CD | | | | | |
| 7 | SBE- | — | 5 | — | 300 | 300 |
| | gamma-CD | | | | | |
| 8 | Alpha-CD | 5 | 5 | 300 | 300 | — |
| 9 | Alpha-CD | — | 5 | — | 300 | 300 |
| 10 | SGM | 2.5 | 2.5 | 300 | 300 | — |

TABLE 2
summary of vinorelbine-CD binding parameters

| | | SBE- | SBE- | | SBE- |
|---|---|---|---|---|---|
| | Alpha- | beta- | gamma- | | alpha- |
| | CD | CD | CD | SGM | CD |
| Binding | 106 | 412 | 121 | 511 | 2137 |
| constant (L/mol) | | | | | |
| ΔbH° (cal/mol) | −1185 | +2305 | +514 | −3177 | −3840 |
| Stoichiometry | 1:1 | 1:1 | 1:1 | 1:1 | 1:1 |

### Example 2: Pharmaceutical Compositions of the Invention

The following compositions were prepared:

TABLE 3

| Ingredients | Quantity % w/v |
|---|---|
| Vinorelbine | 2% |
| Cyclodextrin (SBE-beta-cyclodextrin) | 15.2% |
| Sweetener (Sucralose) | 0.5% |
| Water | QSP |

TABLE 4

| Ingredients | Quantity % w/v |
|---|---|
| Vinorelbine | 2% |
| Cyclodextrin (SBE-beta-cyclodextrin) | 15.2% |
| Sucralose | 1% |
| Maltodextrine | 1% |
| Water | QSP |

The stability composition of Table 4 over 9 months at least after reconstitution was tested and confirmed between 2 and 8° C. See Table 5 below.

TABLE 5

| | | T = 1 | T = 3 | T = 6 | T = 9 |
|---|---|---|---|---|---|
| | T0 | month | months | months | months |
| Vinorelbine | 100.6 ± | 101.8 ± | 103.4 ± | 103.5 ± | 103 ± |
| amount (%) | 1.4 | 1.5 | 0.9 | 2.1 | 3.2 |
| Total | 0.2% | 0.28% | 0.18% | 0.28% | 0.36% |
| impurities (%) | | | | | |

### Example 3: Toxicity Assessment

### 3A—In Vitro Studies

### Materials and Methods

An in vitro study of the toxicity of the vinorelbine-cyclodextrin complex was carried out in cellular models: EpiOral® tissue models provided in a test kit by MatTek® (Brati-slava, Slovak Republic). These models consist of normal human epithelial cells of oral phenotype with morphological and growth characteristics comparable to those obtained in vivo: uniform and reproducible characteristics. After receipt and re-culturing, to allow the tissue to recover further from the stress of shipping and to then measure cytokine release, the tissue was placed in a humidified incubator at 37° C., 5% CO₂ overnight.

Three solutions were prepared: one of vinorelbine 20 mg/ml, one of SBE-CD 305 mg/ml and the last one containing vinorelbine 20 mg/ml and SBE-CD 305 mg/ml.

40 μL of the test solutions were injected into the wells containing the tissue samples. In an initial experiment, the tissue samples were contacted with the solutions for 20 min, 60 min, and 120 min during which the well plates containing the tissues and the solu-tion were placed in the incubator at 37° C., 5% CO2. These times were extended to 1 h, 4 h and 18 h in a second experiment. A 1% triton solution was used as a positive control and water for injection was used as a negative control.

Each condition (tissue, solution and contact time) was triplicated.

The MTT assay for live cell count was performed in each well after the predefined exposure times. At the end of each exposure time and after rinsing with PBS, the inserts were positioned in the labeled wells containing 300 μL of MTT solution and returned to the incubator for 3 hours. Each insert was then removed and immersed in the pre-labeled extraction plate containing 2 mL of the extraction solution. Extraction takes place for 2 hours, in the dark, at room temperature on an orbital shaker. 200 μl of the extraction solution was then transferred to a 96-well microtiter plate to determine the optical density of the extracted samples at 570 and 650 nm. Readings were taken using a Multiskan™ FC microplate photometer supplied by Thermo Scientific™ The calculation formula: % viability=100×[OD (sample)/OD (negative control)] was used to determine the percentage viability for each replicates of the solutions tested.

Assay media from plates incubated before MTT assay were stored in the freezer in labeled vials for subsequent analysis of inflammatory mediators (LDH, PGE-2, IL-1α, IL-1β and other mediators/cytokines).

### Results

The dose-response curve was constructed using a semi-logarithmic scale with percent viability versus dosing time (logarithmic time scale). The ET-50 corresponds to the exposure time at which percent viability has dropped to 50%. In the first experiment, there was a significant difference between vinorelbine alone and vinorelbine/SBE-B-CD complex after 120 minutes of contact time: p-value of 0.034 with a t-test.

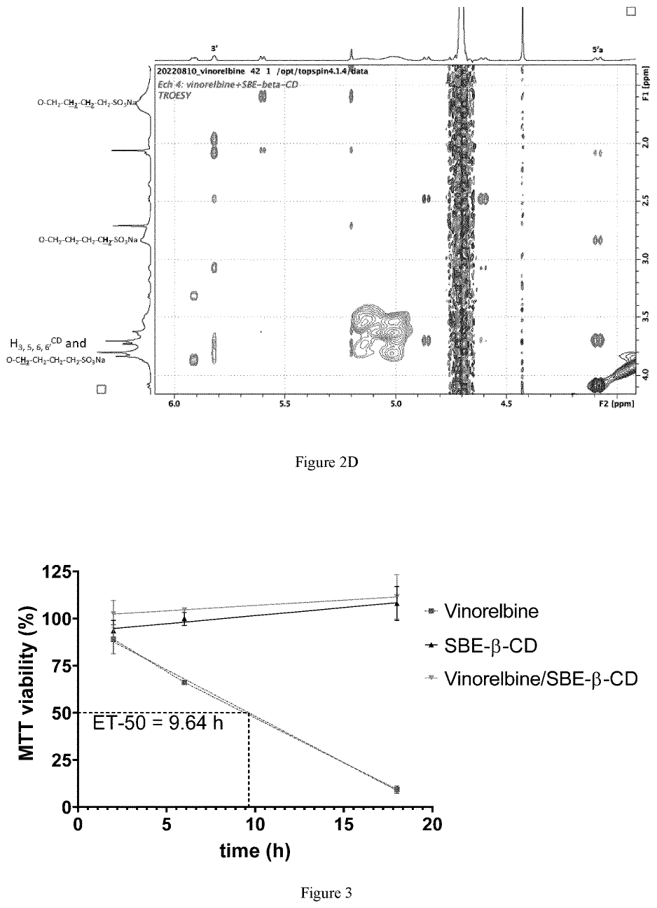

In order to confirm these results, the solution/tissue contact time was prolonged. In the second experiment, ET-50 obtained was about 9.6 h. The results proved that the complexation of vinorelbine in SBE-B-CD clearly masks the toxicity of vinorelbine (see FIG. 3).

### 3B—Immunohistology

### Materials and Methods

EpiOral were also used for immunohistology analysis as follow: 3 untreated tissues (negative control, NC), 3 tissues in contact with SBE-β-CD, 3 tissues in contact with vinorelbine at 20 mg/mL concentration, 3 tissues in contact with vinorelbine complexed with SBE-β-CD and 3 tissues in contact with 1% Triton X-100 (positive control, PC). Tissues samples were fixed with 4% PFA and embedded in paraffin. Sections (3 μm thick) were deparaffinized and were stained with Hematoxylin and Eosin (H&E). For immunofluorescence, slides were incubated overnight at 4° C. with primary antibodies diluted in 0.02% Triton X-100-PBS. Primary antibodies were as follows: rabbit anti-Ki67 (ab16667; 1/75; Abcam), mouse anti-E-Cadherin (ab1416; 1/100; Abcam), rabbit anti-Claudin 4 (ab210796; 1/200; Abcam), mouse anti-Cytokeratin 10 (ab9026; 1/100; Abcam), rabbit anti-Cytokeratin 13 (ab97327; 1/50; Abcam) and mouse anti-Cytokeratin 14 (ab7800; 1/50; Abcam). For immunofluorescence detection, secondary antibody Alexa Fluor™ plus 488 goat anti-mouse (1:200; Invitrogen A11029), Alexa Fluor™ plus 488 donkey anti-rabbit (1:200; Invitrogen A21206), Alexa Fluor™ plus 594 goat anti-mouse (1:200; Invitrogen A32742) and Alexa Fluor™ plus 594 goat anti-rabbit (1:200; Invitrogen A32740) were used.

To analyze apoptotic, autofluorescence was quenched by treating paraffin-embedded sections with PBS/BSA (5%) for 2 h before performing TUNEL staining according to the manufacturer's protocol and using Proteinase K treatment (In Situ Death Detection Kit; Roche). Slides were scanned using NanoZoomer 2.0-RS digital slide scanner (Hamamatsu, Japan). Images were digitally captured from the scanned slides using NDP.view2 software (Hamamatsu).

### Results

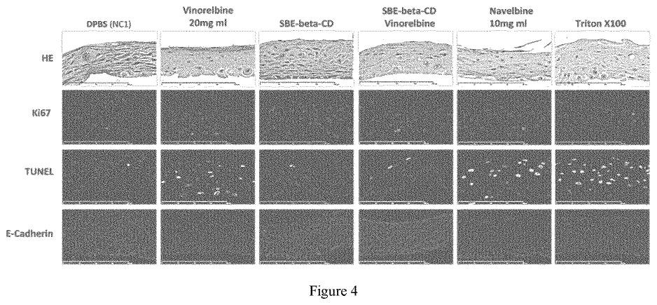

Vinorelbine causes cell mortality and destroys tight tissue junctions. The immunohistology studies showed that complexation with SBE-β-CD reduces the cytotoxicity of vinorelbine, by evidencing a decrease of the TUNEL marking. Moreover we could see a loss of E-cadherin marking when epitheliums are exposed to vinorelbine or Triton, showing a loss of tight junctions. E-cadherin marking is maintained when vinorelbine is trapped (See FIG. 4).

1-15. (canceled)

16. A pharmaceutical composition in oral liquid form, comprising vinorelbine, or a pharmaceutically acceptable salt thereof, and a cyclodextrin that is sulfobutylether-beta-cyclodextrin (SBE-beta-CD).

17. The pharmaceutical composition of claim 16, wherein at least 90% of vinorelbine, or of the pharmaceutically acceptable salt thereof, is in form of an inclusion complex with said cyclodextrin.

18. The pharmaceutical composition of claim 16, wherein the molar ratio of vinorelbine to cyclodextrin is of 1:2 to 1:5.

19. The pharmaceutical composition of claim 16, wherein the pharmaceutically acceptable salt is a ditartrate salt.

20. The pharmaceutical composition of claim 16, which is a ready-to-use solution or is a reconstituted form.

21. The pharmaceutical composition of claim 16, further comprising a sweetener.

22. The pharmaceutical composition of claim 21, wherein the pharmaceutical composition comprises sucralose.

23. The pharmaceutical composition of claim 16, further comprising citric acid.

24. A method for treating a cancer in a patient in need thereof, comprising orally administering the patent with a pharmaceutical composition in oral liquid form, said composition comprising vinorelbine, or a pharmaceutically acceptable salt thereof, and a cyclodextrin that is sulfobutylether-beta-cyclodextrin (SBE-beta-CD).

25. The method of claim 24, wherein the cancer is a rhabdomyosarcoma.

26. The method of claim 24, wherein the patient is a child.

27. An inclusion complex comprising vinorelbine, or a pharmaceutically acceptable salt thereof, and a cyclodextrin that is sulfobutylether-beta-cyclodextrin (SBE-beta-CD).

28. The inclusion complex of claim 27, wherein the molar ratio of vinorelbine to cyclodextrin is of 1:2 to 1:5.

29. The inclusion complex of claim 27, wherein the salt is tartrate salt.

30. A solution which comprises the inclusion complex of claim 27.

31. The solution of claim 30, which is an aqueous solution.

32. The solution of claim 30, wherein at least 90% vinorelbine, or said pharmaceutically acceptable salt thereof, is in form of said inclusion complex with the cyclodextrin.

33. A solid composition, suitable for reconstitution, which composition has been obtained from the solution of claim 30.

34. The composition of claim 33, which is a lyophilized composition.
